(19) United States
(12) Patent Application Publication
(10) Pub. No.: US 2025/0239360 A1
(43) Pub. Date: Jul. 24, 2025

(54) POSITIONING ASSISTING METHOD FOR IMAGE CAPTURE APPARATUS, AND MEDICAL IMAGING SYSTEM

(71) Applicant: GE Precision Healthcare LLC, Waukesha, WI (US)

(72) Inventors: Hao Yang, Beijing (CN); Qingyu Dai, Beijing (CN); Xiaolan Liu, Beijing (CN); Ting Ye, Beijing (CN); Ke Li, Beijing (CN); Zhongyu You, Beijing (CN)

(21) Appl. No.: 19/034,085

(22) Filed: Jan. 22, 2025

(30) Foreign Application Priority Data
Jan. 24, 2024 (CN) 202410098054.X

(51) Int. Cl. G16H 40/60; G06T 7/80; G06V 10/12; G06V 10/22; G16H 30/20

(52) U.S. Cl. CPC G16H 40/60; G06T 7/80; G06V 10/12; G06V 10/22; G16H 30/20

(57) ABSTRACT

A positioning assisting method for an image capture apparatus, and a medical imaging system is provided. The method includes: determining a plurality of image capture apparatus parameters corresponding to a plurality of regions in the medical imaging system; selecting, from the plurality of image capture apparatus parameters, an image capture apparatus parameter corresponding to a region in which a site to be examined of a subject under examination is located; and performing processing of positioning of the site to be examined according to the selected image capture apparatus parameter.

### CROSS REFERENCE TO RELATED APPLICATIONS

The present application claims priority and benefit of Chinese Patent Application No. 202410098054.X filed on Jan. 24, 2024, which is incorporated herein by reference in its entirety.

### TECHNICAL FIELD

Embodiments of the present application relate to the technical field of medical devices, and in particular to a positioning assisting method for an image capture apparatus, and a medical imaging system.

### BACKGROUND

When a magnetic resonance system is used to scan a patient, it is necessary to position a site to be examined (or region of interest) of the patient at a scan center of a magnet of the magnetic resonance system. Typically, the patient to be scanned is placed on a table, and the table is then controlled to move so that the patient on the table and a coil are moved to the scan center of the magnet of the magnetic resonance system for scanning. The table is then moved out after scanning is complete. Current positioning technology mainly uses a positioning light, that is, a positioning light is provided at the entrance of a magnetic resonance scan space. An operator presses a positioning light key on a control panel and moves a table by means of a table movement key on the control panel, so that a site to be examined of the patient and a coil corresponding to the site to be examined are aligned with the positioning light. A landmark key is pressed. The position of the table when aligned is used as a home position. An advance to scan key on the control panel is triggered, so that the table will automatically travel a preset distance and then stop automatically, and the position in which the table stops exactly causes the site to be examined of the patient to be located at a scan center of a magnet of the magnetic resonance system.

### SUMMARY

Provided in embodiments of the present application are a positioning assisting method for an image capture apparatus, and a medical imaging system.

According to an aspect of the embodiments of the present application, a positioning assisting method for an image capture apparatus is provided. The method comprises: determining a plurality of image capture apparatus parameters corresponding to a plurality of regions in the medical imaging system; and selecting, from the plurality of image capture apparatus parameters, an image capture apparatus parameter corresponding to a region in which a site to be examined of a subject under examination is located. The method also includes performing processing of positioning of the site to be examined according to the selected image capture apparatus parameter.

According to an aspect of the embodiments of the present application, a computer-readable storage medium is provided. The computer-readable storage medium comprises a stored computer program. The positioning assisting for an image capture apparatus described in the foregoing aspect is performed when the computer program is run.

According to one aspect of the embodiments of the present application, a medical imaging system is provided. The system comprises: an image capture apparatus, for capturing image data; and a controller, connected to the image capture apparatus and configured to perform the positioning assisting method for an image capture apparatus in the foregoing aspect.

With reference to the following description and drawings, specific implementations of the embodiments of the present application are disclosed in detail, and the means by which the principles of the embodiments of the present application can be employed are illustrated. It should be understood that the embodiments of the present application are not limited in scope thereby. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of coordinate systems according to an embodiment of the present application;

FIG. 2 is a diagram of an example of coordinate conversion between a world coordinate system and an image capture apparatus coordinate system according to an embodiment of the present application;

FIG. 3 is a diagram of an example of coordinate conversion between an image capture apparatus coordinate system and an image coordinate system according to an embodiment of the present application;

FIG. 4 is a diagram of an example of coordinate conversion between an image coordinate system and a pixel coordinate system according to an embodiment of the present application;

FIG. 5 is a schematic diagram of a magnetic resonance imaging system according to an embodiment of the present application;

FIG. 6 is a schematic diagram of a positioning assisting method for an image capture apparatus according to an embodiment of the present application;

FIG. 7 is a schematic diagram of an implementation of operation 601 according to an embodiment of the present application;

FIG. 8 is a schematic diagram of M calibration positions according to an embodiment of the present application;

FIG. 9 is a schematic diagram of M acquired images according to an embodiment of the present application;

FIG. 10 is a schematic diagram of an image capture apparatus parameter checking method according to an embodiment of the present application;

FIG. 11 is a schematic diagram of an implementation of operation 602 according to an embodiment of the present application;

FIG. 12 is a schematic diagram of an implementation of operation 603 according to an embodiment of the present application;

FIG. 13 is a schematic diagram of a medical imaging system according to an embodiment of the present application;

FIG. 14 is a schematic diagram of a magnetic resonance imaging system according to an embodiment of the present application;

FIG. 15 is a schematic diagram of an implementation of operations 701 to 703 according to an embodiment of the present application; and

FIG. 16 is a schematic diagram of a positioning assisting apparatus for an image capture apparatus according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

To facilitate understanding, some technical terms involved in the embodiments of the present application will be described below first.

According to an imaging principle of an image capture apparatus (e.g., a camera), in order to determine a geometric relationship corresponding to a certain point on a surface of a captured object on an image capture apparatus plane, a geometric parameter model of the camera needs to be established. The geometric parameters include external parameters and internal parameters. The internal parameters are related to inherent characteristics of the image capture apparatus such as a focal length, a pixel size, a distortion shape, and the like. The external parameters are used to describe a relationship between an image capture apparatus coordinate system and a world coordinate system. The internal and external parameters of the camera need to be calculated via corresponding algorithms. A procedure of acquiring the parameters is referred to as calibration of the image capture apparatus.

The world coordinate system refers to an absolute coordinate system of an objective three-dimensional world, and may also be referred to as an objective coordinate system. For example, a reference coordinate system is selected in an environment to describe the positions of the image capture apparatus and the object, and the coordinate system is a world coordinate system. The image capture apparatus coordinate system is a coordinate system established by using an image capture apparatus spot as the center, the X and Y axes being parallel to two sides of an image, and the optical axis being the Z axis. An image coordinate system is typically an image coordinate system of an imaging plane, and the origin is a midpoint of the imaging plane. In a pixel coordinate system, the origin is an upper left corner of an image, and the unit of coordinate points in the pixel coordinate system is a pixel.

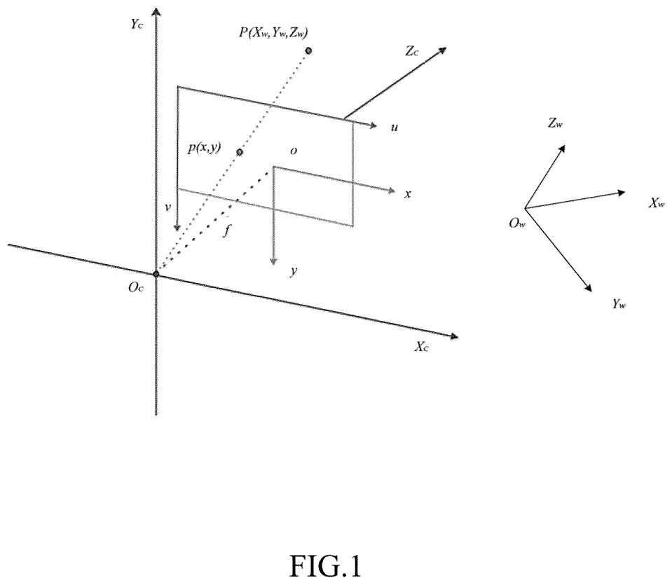

FIG. 1 is a schematic diagram of coordinate systems according to an embodiment of the present application. For example, it is assumed that coordinates of an object P in a world coordinate system are (X_{w}, Y_{w}, Z_{w}), and the origin is O_{w}. Coordinates in an image capture apparatus coordinate system are (X_{c}, Y_{c}, Z_{c}), and the origin is O_{c}. p is an imaging point of the object P in an image, and coordinates thereof in an image coordinate system are (x, y). The origin is o. Coordinates of p in a pixel coordinate system are (u, v). Furthermore, f is a focal length of a camera, is equal to a distance between o and O_{c}, and may be expressed by formula (1):

$$f = ‖ o - {0}_{c} ‖ \qquad ( 1 )$$

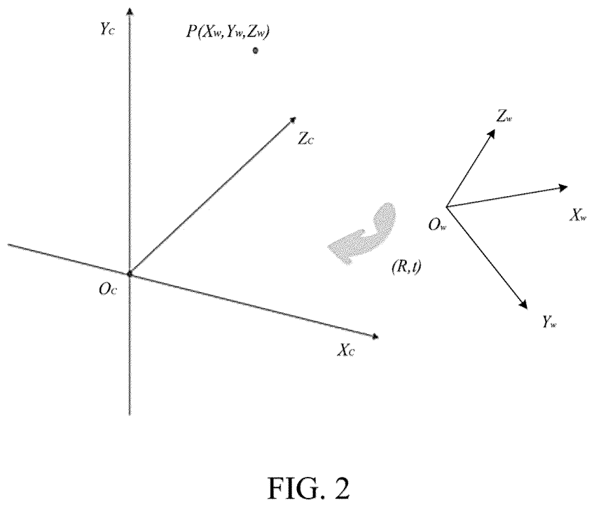

FIG. 2 is a diagram of an example of coordinate conversion between a world coordinate system and an image capture apparatus coordinate system according to an embodiment of the present application. A certain point in the world coordinate system may be converted into a point in the image capture apparatus coordinate system via rotation (a rotation matrix R) and translation (a translation vector t). The coordinates of the point P in the image capture apparatus coordinate system may be acquired on the basis of the coordinates in the world coordinate system, and may be expressed by, for example, formula (2):

$$[ \begin{matrix}{X}_{c} \\ {Y}_{c} \\ {Z}_{c}\end{matrix} ] = R [ \begin{matrix}{X}_{w} \\ {Y}_{w} \\ {Z}_{w}\end{matrix} ] + t \qquad ( 2 )$$

Furthermore, the coordinates of the point P in the world coordinate system may also be converted into coordinates in the image capture apparatus coordinate system via homogeneous coordinates, which may be expressed by, for example, formula (3):

$$[ \begin{matrix}{X}_{c} \\ {Y}_{c} \\ {Z}_{c} \\ 1\end{matrix} ] = [ \begin{matrix}R & t \\ 0 & 1\end{matrix} ] [ \begin{matrix}{X}_{w} \\ {Y}_{w} \\ {Z}_{w} \\ 1\end{matrix} ] \qquad ( 3 )$$

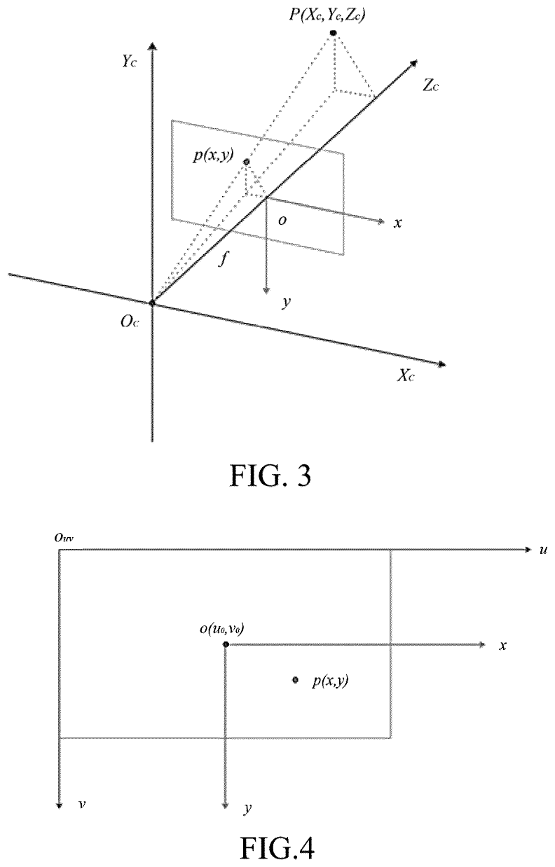

FIG. 3 is a diagram of an example of coordinate conversion between an image capture apparatus coordinate system and an image coordinate system according to an embodiment of the present application. The conversion of the image capture apparatus coordinate system into the image coordinate system is a perspective projection relationship. As shown in FIG. 3, after the coordinates of the object in the image capture apparatus coordinate system are acquired, the coordinate system of the object may be further converted from the three-dimensional image capture apparatus coordinate system into the two-dimensional image coordinate system, which may be expressed by, for example, formula (4):

$${Z}_{c} [ \begin{matrix}x \\ y \\ 1\end{matrix} ] = [ \begin{matrix}f & 0 & 0 & 0 \\ 0 & f & 0 & 0 \\ 0 & 0 & 1 & 0\end{matrix} ] [ \begin{matrix}{X}_{c} \\ {Y}_{c} \\ {Z}_{c} \\ 1\end{matrix} ] \qquad ( 4 )$$

FIG. 4 is a diagram of an example of coordinate conversion between an image coordinate system and a pixel coordinate system according to an embodiment of the present application. For example, the image coordinate system and the pixel coordinate system are both two-dimensional coordinate systems on the imaging plane, but have different origins and measurement units. As shown in FIG. 4, the origin of the image coordinate system is an intersection of the optical axis of the camera and the imaging plane, and is typically a midpoint or a principle point of the imaging plane. Furthermore, the unit of the image coordinate system is millimeter, which is a physical unit. However, the unit of the pixel coordinate system is pixel, and a pixel is expressed by a certain row and a certain column, so that dx and dy are used to indicate how many millimeters there are in each column and each row. The conversion of the image coordinate system into the pixel coordinate system may be expressed by formula (5):

$$[ \begin{matrix}u \\ v \\ 1\end{matrix} ] = [ \begin{matrix}\frac{1}{dx} & 0 & {u}_{0} \\ 0 & \frac{1}{dy} & {v}_{0} \\ 0 & 0 & 1\end{matrix} ] [ \begin{matrix}x \\ y \\ 1\end{matrix} ] \qquad ( 5 )$$

In this way, coordinates of an object in the world coordinate system may be converted into coordinates in the pixel coordinate system via the above conversion between the coordinate systems, which may be expressed by, for example, formula (6):

$$\begin{matrix}{Z}_{c} [ \begin{matrix}u \\ v \\ 1\end{matrix} ] = [ \begin{matrix}\frac{1}{dx} & 0 & {u}_{0} \\ 0 & \frac{1}{dy} & {v}_{0} \\ 0 & 0 & 1\end{matrix} ] [ \begin{matrix}f & 0 & 0 & 0 \\ 0 & f & 0 & 0 \\ 0 & 0 & 1 & 0\end{matrix} ] [ \begin{matrix}R & t \\ 0 & 1\end{matrix} ] [ \begin{matrix}{X}_{w} \\ {Y}_{w} \\ {Z}_{w} \\ 1\end{matrix} ] \\ = [ \begin{matrix}{f}_{x} & 0 & {u}_{0} & 0 \\ 0 & {f}_{y} & {v}_{0} & 0 \\ 0 & 0 & 1 & 0\end{matrix} ] [ \begin{matrix}R & t \\ 0 & 1\end{matrix} ] [ \begin{matrix}{X}_{w} \\ {Y}_{w} \\ {Z}_{w} \\ 1\end{matrix} ]\end{matrix} \qquad ( 6 )$$

Image capture apparatus parameters include at least internal parameters and external parameters. The internal parameters include a distance (i.e., a focal length f) between a physical imaging plane and an optical center. The external parameters include the rotation matrix R and the translation vector t of the image capture apparatus coordinate system and the world coordinate system.

Therefore, the matrix

$$[ \begin{matrix}{f}_{x} & 0 & {u}_{0} & 0 \\ 0 & {f}_{y} & {v}_{0} & 0 \\ 0 & 0 & 1 & 0\end{matrix} ]$$

may also be referred to as an internal parameter matrix. The matrix

$$[ \begin{matrix}R & t \\ 0 & 1\end{matrix} ]$$

may also be referred to as an external parameter matrix.

In addition, the coordinates of the object in the pixel coordinate system may also be converted into the coordinates in the world coordinate system via an inverse process of formula (6) and the above parameters such as the internal parameter matrix and the external parameter matrix.

In medical imaging systems, an image capture apparatus is increasingly used as an auxiliary tool to acquire patient information to simplify scanning workflow. If an image capture apparatus is applied to a medical imaging system, in addition to the above calibration of the internal and external parameters, the image capture apparatus coordinate system needs to be further mapped to a medical imaging system coordinate system. For example, in a table coordinate system, a corresponding coordinate conversion relationship needs to be further calculated. Similar to formula (2), a certain point in the medical imaging coordinate system may be converted into a point in the image capture apparatus coordinate system via rotation (a rotation matrix R′) and translation (a translation vector t′). The matrix

$$[ \begin{matrix}{R}^{′} & {t}^{′} \\ 0 & 1\end{matrix} ]$$

may also be referred to as a conversion matrix between the image capture apparatus coordinate system and the medical imaging system coordinate system, and when

$$[ \begin{matrix}R & t \\ 0 & 1\end{matrix} ]$$

in formula (6) is replaced with

$$[ \begin{matrix}{R}^{′} & {t}^{′} \\ 0 & 1\end{matrix} ] ,$$

a coordinate conversion relationship between the medical imaging coordinate system and the pixel coordinate system can be acquired.

Currently, in the related art, automatic positioning for scanning may be performed by using an image capture apparatus instead of a positioning light. The inventors have found that the accuracy of the automatic positioning performed by using the image capture apparatus is affected by at least calibration precision of the image capture apparatus. The image capture apparatus is typically provided at a ceiling above a table, and the accuracy is high only when calibration calculation is performed in some regions of the table facing the image capture apparatus, but there is a large error when calibration calculation is performed on other regions of the table. In addition, a calibration procedure relies on a plurality of manual operations performed by an operator, and the calibration procedure is prone to error.

In response to at least one of the above problems, embodiments of the present application provide a positioning assisting method for an image capture apparatus, and a medical imaging system. A plurality of image capture apparatus parameters corresponding to a plurality of regions in the medical imaging system are determined, and one image capture apparatus parameter is selected from the plurality of image capture apparatus parameters to perform coordinate conversion, so that image capture apparatus calibration precision in all positions on a table can be ensured, thereby improving accuracy of automatic positioning prediction. In addition, the entire calibration procedure is automatically performed, thereby avoiding human operation errors and simplifying a scanning procedure.

A medical imaging system described herein includes, but is not limited to, a computed tomography (CT) system, a magnetic resonance imaging (MRI) system, a C-arm imaging system, a positron emission computed tomography (PET) system, a single photon emission computed tomography (SPECT) system, an ultrasonic system, an X-ray imaging system, or any other suitable medical imaging system.

In the following, a magnetic resonance imaging system is used as an example for description, but embodiments of the present application are not limited thereto.

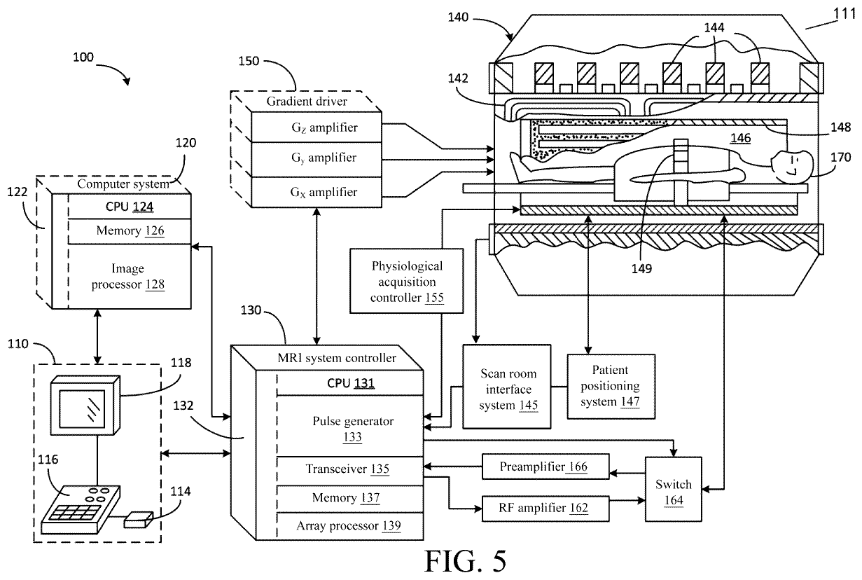

For ease of understanding, FIG. 5 shows a magnetic resonance imaging (MRI) system 100 according to some embodiments of the present invention.

The MRI system 100 includes a scanning unit 111. The scanning unit 111 is configured to perform a magnetic resonance scan on a subject (for example, a human body) 170 to generate a reconstructed image of a region of interest of the subject 170. The region of interest may be a predetermined anatomical site or anatomical tissue.

The operation of the MRI system 100 is controlled by an operator workstation 110 that includes an input device 114, a control panel 116, and a display 118. The input apparatus 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and in communication with a computer system 120 that enables an operator to control the generation and display of images on the display 118. The computer system 120 includes various components that communicate with one another via an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by image processing functions implemented in the CPU 124. The computer system 120 may be connected to an archive media device, a persistent or backup memory, or a network. The computer system 120 may be coupled to and communicates with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator 133 which is in communication with the operator workstation 110, a transceiver (or an RF transceiver) 135, a memory 137, and an array processor 139. In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be performed during an MRI scan, so as to be used to control the scanning unit 111 to perform the flow of the aforementioned magnetic resonance scan. The MRI system controller 130 is further coupled to and in communication with a gradient driver system 150, which is coupled to a gradient coil assembly 142 to generate a magnetic field gradient during the MRI scan.

The sequence pulse generator 133 may further receive data from a physiological acquisition controller 155, which receives signals from a number of different sensors, such as electrocardiogram (ECG) signals from electrodes attached to a patient, which are connected to the subject or patient 170 undergoing an MRI scan. The sequence pulse generator 133 is coupled to and in communication with a scan room interface system 145 that receives signals from various sensors associated with the state of the resonance assembly 140. The scan room interface system 145 is further coupled to and in communication with a patient positioning system 147 that sends and receives signals to control movement of a patient table to a desired position to perform the MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field B₀ throughout a cylindrical imaging volume 146. The resonance assembly 140 further includes an RF body coil 148, which, in operation, provides a transverse magnetic field B₁, the transverse magnetic field B₁ being substantially perpendicular to B₀ throughout the entire cylindrical imaging volume 146. The resonance assembly 140 may further include an RF surface coil 149 for imaging different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in the k-space. The y direction may be referred to as a phase encoding direction or a k_{y} direction in the k-space. Gₓ can be used for frequency encoding or signal readout, and is generally referred to as a frequency encoding gradient or a readout gradient. G_{y} can be used for phase encoding, and is generally referred to as a phase encoding gradient. G_{z} can be used for slice (layer) position selection to acquire k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses that are amplified by an RF amplifier 162 and provided to the RF body coil 148 through a transmit/receive switch (T/R switch) 164.

As described above, the RF body coil 148 and the RF surface coil 149 may be used to transmit RF excitation pulses and/or receive resulting MR signals from the patient undergoing the MRI scan. The MR signals emitted by excited nuclei in the patient of the MRI scan may be sensed and received by the RF body coil 148 or the RF surface coil 149 and sent back to a preamplifier 166 through the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be acquired by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image to be reconstructed, the data is rearranged into separate k-space data arrays, and each of the separate k-space data arrays is input into the array processor 139, and the array processor is operated to transform the data into an array of reconstructed images by Fourier transform.

The array processor 139 uses transform methods, most commonly Fourier transform, to create images from the received MR signals. These images are transmitted to the computer system 120 and stored in the memory 126. In response to commands received from the operator workstation 110, data for the reconstructed image may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 5 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program used to implement scanning processing (such as a scan flow and an imaging sequence), image reconstruction, image processing, etc. For example, the storage medium may store a program used to implement the magnetic resonance imaging method according to the embodiments of the present invention. The described storage medium may include, for example, a ROM, a floppy disk, a hard disk, an optical disk, a magneto-optical disk, a CD-ROM, or a non-volatile memory card.

The aforementioned “imaging sequence” (also referred to below as a scanning sequence or a pulse sequence) refers to a combination of pulses having specific amplitudes, widths, directions, and time sequences and applied when a magnetic resonance imaging scan is executed. These pulses may typically include, for example, radio-frequency pulses and gradient pulses. The radio-frequency pulses may include, for example, radio-frequency excitation pulses, radio-frequency refocusing pulses, inverse recovery pulses, etc. The gradient pulses may include, for example, the aforementioned gradient pulse used for layer selection, gradient pulse used for phase encoding, gradient pulse used for frequency encoding, gradient pulse used for phase shifting (phase shift), gradient pulse used for dispersion of phases (dephasing), etc. Typically, a plurality of scan sequences can be preset in the magnetic resonance system, so that the sequence suitable for clinical detection requirements can be selected. The clinical detection requirements may include, for example, an imaging site, an imaging function, an imaging effect, and the like.

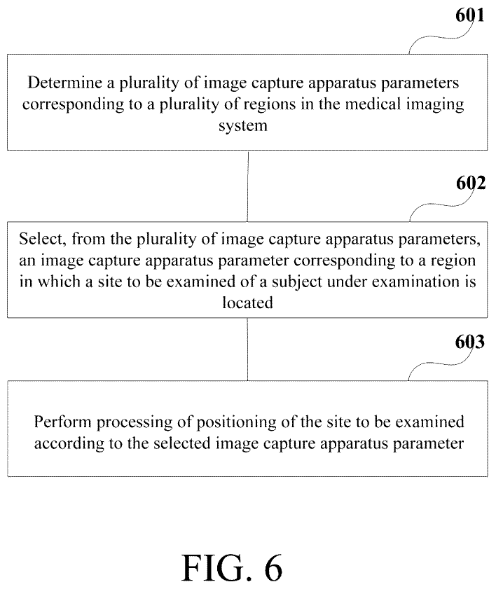

A positioning assisting method for an image capture apparatus according to an embodiment of the present application will be described below with reference to the embodiment. FIG. 6 is a schematic diagram of a positioning assisting method for an image capture apparatus according to an embodiment of the present application. As shown in FIG. 6, the method includes: determining a plurality of image capture apparatus parameters corresponding to a plurality of regions in the medical imaging system at 601. At step 602, the method includes selecting, from the plurality of image capture apparatus parameters, an image capture apparatus parameter corresponding to a region in which a site to be examined of a subject under examination is located. At step 603, the method includes performing processing of positioning of the site to be examined according to the selected image capture apparatus parameter.

In some embodiments, the plurality of (hereinafter referred to as M, M being an integer greater than or equal to 2) regions in the medical imaging system do not overlap with each other, and the M regions are the same or different in size. For example, when the medical imaging system is a magnetic resonance imaging system, the plurality of regions may be different regions in a superior-inferior (SI) direction (e.g., a z direction), or different regions in an anterior-posterior (AP) direction, or different regions in a right-left (RL) direction, and the embodiments of the present application are not limited thereto. In the following embodiments, the SI direction is used as an example for ease of description.

In some embodiments, each of the M regions may be represented by a range of coordinate positions. The coordinate position is in a certain direction in the medical imaging system coordinate system. For example, when the medical imaging system coordinate system is a table coordinate system, the table may move in the horizontal (i.e., SI) direction, or may move in the height (i.e., AP) direction, and the M regions may be represented by ranges of coordinate positions in the table movement direction. The table is for carrying a scan subject, e.g., a subject under examination, and is configured to be capable of communicating with an entrance of a scanning chamber of the medical imaging system. For example, the table may move from the outside of the scanning chamber into the inside of the scanning chamber, and vice versa.

For example, in the SI direction, the coordinate position of the rear of the table, or the front of the table, or the middle of the table may be used as the coordinate position of the table. A coordinate of a home position of the table is 0. The table can move to a scan center. A coordinate of a farthest position to which the table can move is 2000. The M regions may be represented as [100, 300], (300, 600], (600, 900], (900, 1200], (1200, 1500], and (1500, 1800], which are merely examples, and the embodiments of the present application are not limited thereto. The value of M may be determined as desired, and will be specifically described in the following embodiments.

In some embodiments, the image capture apparatus parameter includes a conversion matrix between an image capture apparatus coordinate system and the medical imaging system coordinate system. The conversion matrix may be a conversion matrix of conversion from the image capture apparatus coordinate system to the medical imaging system coordinate system, or a conversion matrix of conversion from the medical imaging system coordinate system to the image capture apparatus coordinate system. For example, the conversion matrix is represented by

$$[ \begin{matrix}{R}^{′} & {t}^{′} \\ 0 & 1\end{matrix} ] .$$

In some embodiments, in 601, the plurality of regions are in a one-to-one correspondence with the plurality of image capture apparatus parameters. That is, for each of the M regions, the image capture apparatus parameter corresponding to the region is separately determined. That is, M image capture apparatus parameters are determined for the M regions. Details will be described below.

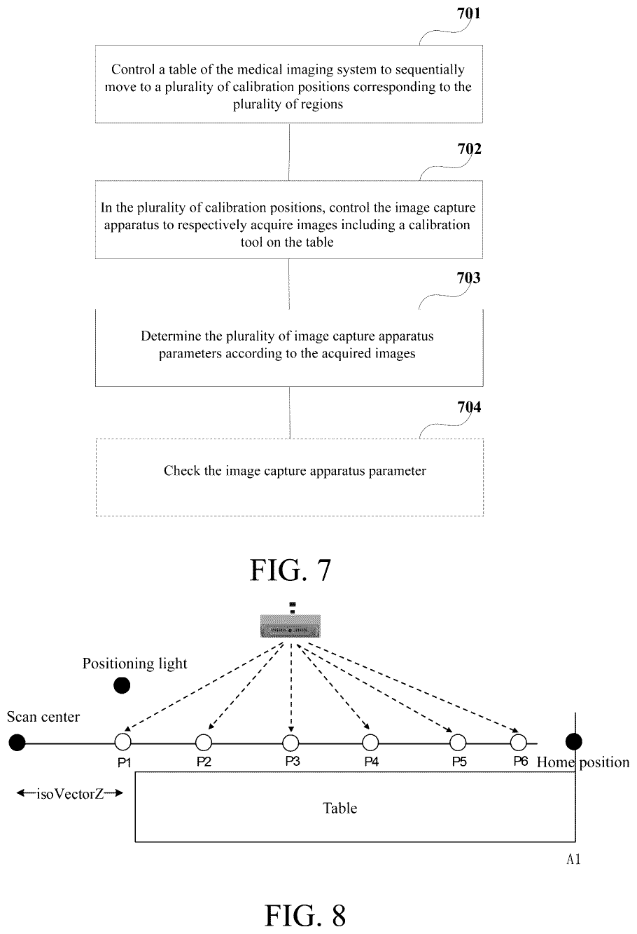

FIG. 7 is a schematic diagram of an implementation of operation 601 of the embodiments of the present application. As shown in FIG. 7, operation 601 includes: a step 701 of controlling a table of the medical imaging system to sequentially move to a plurality of calibration positions corresponding to the plurality of regions. At step 702, in the plurality of calibration positions, the image capture apparatus is controlled to respectively acquire images including a calibration tool on the table; and step 703, the plurality of image capture apparatus parameters are determined according to the acquired images.

In some embodiments, in 701, the plurality of calibration positions are in a one-to-one correspondence with the plurality of regions. The calibration position may correspond to a middle position or an edge position of the region, and the embodiments of the present application are not limited thereto. The calibration position may be represented by a coordinate in a certain direction in the table coordinate system. Distances between adjacent calibration positions among the plurality of calibration positions are the same.

FIG. 8 is a schematic diagram of a plurality of calibration positions according to an embodiment of the present application. As shown in FIG. 8, the table is in an initial position, and P1, P2, P3, P4, P5, and P6 are a plurality of calibration positions for movement of the table. Description is provided below by using an example in which P1, P2, P3, P4, P5, and P6 are coordinate positions of an edge A1 of the table in the SI direction. P1 is used as an initial calibration position, and P2, P3, P4, P5, and P6 are sequentially used as next calibration positions. That is, the table is controlled to sequentially move to the positions P1, P2, P3, P4, P5, and P6 and stop. Alternatively, P6 may be used as an initial position, and the table is controlled to sequentially move to the positions P6, P5, P4, P3, P2, and P1 and stop. The embodiments of the present application are not limited thereto, and description is provided below by using an example in which P1 is the initial calibration position.

How to determine the plurality of calibration positions will be described below.

In some embodiments, a coordinate of the initial calibration position P1 may be determined first. The initial calibration position may be predefined, or the initial calibration position for movement of the table may be determined according to a maximum distance that the table can move and a distance between a positioning light and the scan center in the medical imaging system. For example, the initial calibration position is a difference between the maximum distance and the distance between the positioning light and the scan center.

In the following, the coordinate position in the SI direction in a sickbed coordinate system is used as an example. The distance between the positioning light and the scan center (the isocenter of the magnetic resonance imaging system) is isoVectorZ (in the medical imaging system coordinate system). The maximum distance that the table can move is tableLimit (in the medical imaging system coordinate system). The initial calibration position is P1=tableLimit −isoVectorZ. The initial calibration position is a position that can be scanned by the positioning light.

In some embodiments, after the initial calibration position is determined, the positioning light of the medical imaging system is turned on, and the calibration tool on the table is aligned with the positioning light. The calibration tool may be placed on the table in advance, or may be placed on the table after the table moves to the initial calibration position, and the embodiments of the present application are not limited thereto.

For example, the calibration tool may be a checkerboard, so that image capture apparatus calibration is performed by using a checkerboard calibration method, but the embodiments of the present application are not limited thereto. The calibration tool may also be a pattern of uniformly distributed or regularly distributed dots or the like. Alternatively, the calibration tool may be an image including parallel lines, and the parallel lines may be walls in a building, lane lines on a road, or the like, so that image capture apparatus calibration is performed by using a vanishing point method. Examples are not enumerated herein. The aligning the calibration tool with the positioning light includes keeping the calibration tool horizontal and aligning a horizontal laser line of the positioning light with the calibration tool. For example, when the calibration tool is a checkerboard, the horizontal laser line of the positioning light is aligned with an edge of a square in the checkerboard.

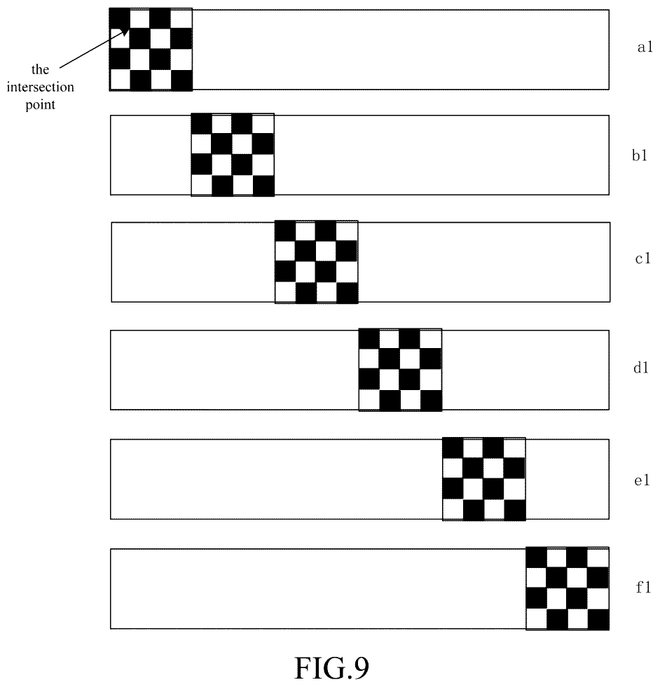

In some embodiments, a distance of each movement of the table and the number of calibration positions may be determined according to a size of the calibration tool and a length of the table, so as to determine calibration positions among the plurality of calibration positions other than the initial calibration position. The size of the calibration tool in a lengthwise direction of the table is L (in the medical imaging system coordinate system). The distance of each movement of the table is the size L of the calibration tool in the lengthwise direction of the table. That is, the distance between adjacent calibration positions is L. The number of calibration positions is M=┌the length of the table÷L┐. That is, coordinate positions of the M calibration positions are respectively P1, P1−L, P1−2L, P1−3L, P1−4L, . . . , P1−(M−1)L. The M regions corresponding to the M calibration positions may be (P1−L, P], (P1−2L, P−L], (P1−3L, P1−2L], . . . , (P1−(M−2)L, P1−(M−1)L], which are merely illustrative, and the embodiments of the present application are not limited thereto. For example, the M regions may also be (P1−L+L/2, P1+L/2], (P1−2L+L/2, P1−L+L/2], (P1−3L+L/2, P1−2L+L/2], . . . , (P1−(M−2)L+L/2, P1−(M−1)L+L/2]. In some embodiments, in 702, in the plurality of calibration positions, the image capture apparatus is controlled to respectively acquire images including a calibration tool on the table, and the images acquired by the image capture apparatus are acquired. The image capture apparatus may be a 3D camera or a 2D optical camera, and the embodiments of the present application are not limited thereto. The image capture apparatus can capture an image within an angle of view thereof in real time. The image data may include two-dimensional optical image data (a two-dimensional RGB image), and optionally may further include depth image data, but is not limited thereto. For example, the image capture apparatus, i.e., the 3D camera, may capture images in real time, generate a video stream, and display the same on a graphical user interface of the medical imaging system in real time. The image capture apparatus may be mounted at a ceiling or a wall directly above an examination bed of a medical imaging system. Embodiments of the present application are not limited thereto. In the M calibration positions, the image capture apparatus is controlled to respectively acquire images including the calibration tool on the table. In the acquired images, the position (pixel coordinates) of the table changes, and the position (pixel coordinates) of the calibration tool changes, but the position of the calibration tool relative to the table does not change. FIG. 9 is a schematic diagram of M acquired images according to an embodiment of the present application. As shown in FIG. 9, in the example in which the calibration tool is a checkerboard, a1 to f1 are respectively images corresponding to the respective calibration positions in FIG. 8. For example, a pixel range of the image in a widthwise direction is 0 to 180, and pixel coordinate ranges of the checkerboard in the images corresponding to the respective calibration positions are respectively [0, 30], (30, 60], (60, 90], (90, 120], (120, 150], and (150, 180].

In some embodiments, in 703, corresponding image capture apparatus parameters are respectively determined for the respective images. The image capture apparatus parameter may be determined according to the acquired image by using a checkerboard method, or a vanishing point based method, or the like. For details, reference may be made to the related art. A checkerboard algorithm is used as an example. In a world coordinate system, a distance between intersection points of black and white squares in the checkerboard is known. A pixel coordinate of an intersection point of black and white squares in the predetermined checkerboard (as shown in FIG. 9) in the acquired image is determined first, and a coordinate of the intersection point of the black and white squares in the predetermined checkerboard in the image capture apparatus coordinate system can then be acquired with reference to known external parameters and internal parameters (for example, set during factory setting or calibrated) of the image capture apparatus and formulas (1) to (6) described above. A coordinate position of the intersection point of the black and white squares in the predetermined checkerboard in the medical imaging system coordinate system is also known (the coordinate position of the table corresponding to the image is known, and the position of the checkerboard relative to the table is known). Therefore, the conversion matrix between the image capture apparatus coordinate system and the medical imaging system coordinate system can be determined on the basis of the coordinate of the intersection point of the black and white squares in the predetermined checkerboard in the medical imaging system coordinate system and the coordinate of the intersection point of the black and white squares in the predetermined checkerboard in the image capture apparatus coordinate system.

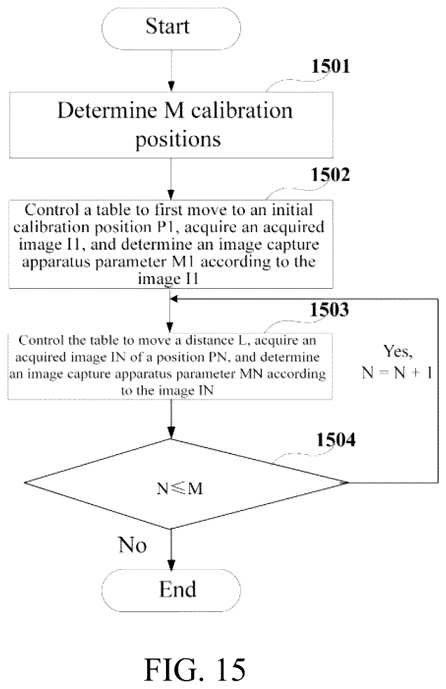

FIG. 15 is a schematic diagram of an implementation of operations 701 to 703 according to an embodiment of the present application. As shown in FIG. 15, the method includes: a step 1501 for determining M calibration positions; and step 1502 for controlling the table to first move to an initial calibration position P1, aligning the calibration tool on the table with a positioning light, controlling the image capture apparatus to acquire an image I1 of the initial calibration position, acquiring the acquired image I1, and determining an image capture apparatus parameter M1 corresponding to a region corresponding to the initial calibration position P1 according to the image I1 (pixel coordinates of an intersection point). The method also includes a step 1503 for controlling the table to move a distance L (in a medical imaging coordinate system), that is, moving the table to PN, controlling the image capture apparatus to acquire an image IN of the position PN, acquiring the acquired image IN, and determining an image capture apparatus parameter MN (the initial value of N is 2) corresponding to a region corresponding to the position PN according to the image IN. At step 1504, it is determined whether N is less than or equal to M, and when a determination result is yes, returning to step 1503, and N=N+1, or otherwise, ending the operation.

In some embodiments, in order to improve image capture apparatus calibration precision, optionally, the method may further include: 704, checking the image capture apparatus parameters.

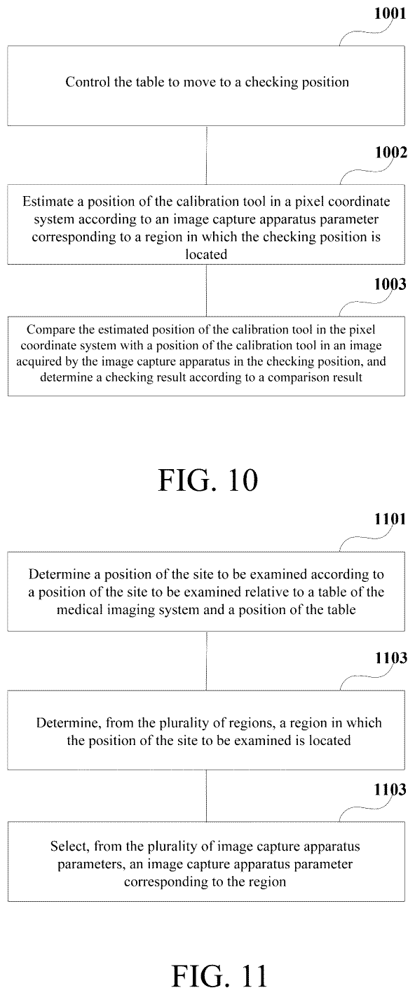

FIG. 10 is a schematic diagram of an image capture apparatus parameter checking method according to an embodiment of the present application. As shown in FIG. 10, the method includes: a step 1001 for controlling the table to move to a checking position; a step 1002 for estimating a position of the calibration tool in a pixel coordinate system according to an image capture apparatus parameter corresponding to a region in which the checking position is located. At step 1003, the estimated position of the calibration tool in the pixel coordinate system is compared with a position of the calibration tool in an image acquired by the image capture apparatus in the checking position, and determining a checking result according to a comparison result.

In some embodiments, the checking position P′ may be any position (except the aforementioned calibration position in the medical imaging system coordinate system) where any table can stop, and the embodiments of the present application are not limited thereto. There may be one or more checking positions, and the checking position may be represented in the same manner as the calibration position. A coordinate of the checking position is compared with coordinate ranges of the M regions to determine a region in which the coordinate of the checking position is, and an image capture apparatus parameter M′ corresponding to the region is selected. The image capture apparatus is controlled to acquire an image I′ corresponding to the checking position P′. A coordinate X1 of the pixel coordinate system corresponding to an intersection point of black and white squares in the predetermined checkerboard is determined in the image I′. A coordinate in the image capture apparatus coordinate system corresponding to the intersection point of the black and white squares in the predetermined checkerboard is calculated according to the image capture apparatus parameter M′ and the coordinate (in the medical imaging system coordinate system) of the checking position P′, and a coordinate X2 in the pixel coordinate system corresponding to the intersection point of the black and white squares in the predetermined checkerboard is estimated with reference to the internal parameters and the external parameters of the image capture apparatus and formulas (1) to (6) described above. A difference between the coordinates X1 and X2 is calculated, and a checking result is determined according to the value of the difference between X1 and X2. For example, if the difference is less than or equal to two pixels, it is indicated that precision is high, and processing may be ended; otherwise, it is indicated that the precision is low, 701 needs to be performed to re-determine the image capture apparatus parameters.

In some embodiments, when scanning a site to be examined of a subject under examination, the table no longer carries any calibration tool, but carries the subject under examination, the site to be examined of the subject under examination may be provided with a coil. Before the scanning is formally started, positioning needs to be performed. That is, the table needs to be moved, and the scanning can be performed only when the site to be examined (i.e., a center position of the coil covering the site to be examined) is aligned with the scan center of the medical imaging system. How to determine a distance moved by the table will be described in detail below.

In some embodiments, FIG. 11 is a schematic diagram of an implementation of operation 602 according to an embodiment of the present application. As shown in FIG. 11, operation 602 includes: a step 1101 for determining a position of the site to be examined according to a position of the site to be examined relative to a table of the medical imaging system and a position of the table. Operation 602 also includes a step 1102 for determining, from the plurality of regions, a region in which the position of the site to be examined is located; and a step 1103 for selecting, from the plurality of image capture apparatus parameters, an image capture apparatus parameter corresponding to the region.

In some embodiments, in 1101, a coordinate position of the site to be examined in the medical imaging system coordinate system may be calculated according to the corresponding position of the site to be examined on the table and the coordinate position of the table. It is assumed that a coordinate position P″ of the edge A1 of the table in the SI direction is the position of the table, and a distance between the site to be examined and the edge A1 of the table may be determined as Y (in the medical imaging system coordinate system) according to a ruler, so that the coordinate position of the site to be examined is P″+Y (in the medical imaging system coordinate system). The position P″ of the table may be the home position of the table or any predefined position, and the embodiments of the present application are not limited thereto. In 1102 and 1103, the coordinate of the site to be examined is compared with the plurality of regions in 601 to determine the region in which the coordinate of the site to be examined is located, and an image capture apparatus parameter M″ corresponding to the region is selected from the M image capture apparatus parameters.

In some embodiments, mutual conversion between coordinate positions in the medical imaging coordinate system and coordinate positions in the image capture apparatus coordinate system may be performed according to the image capture apparatus parameter M″, so as to perform processing of corresponding positioning.

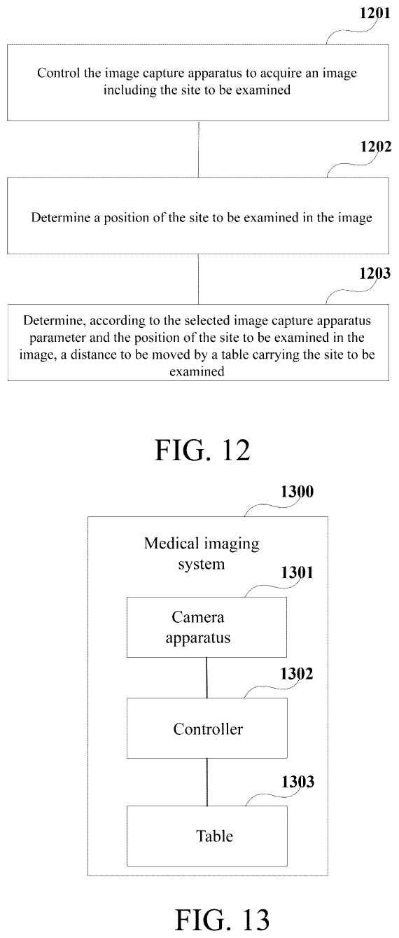

In some embodiments, FIG. 12 is a schematic diagram of operation 603 according to an embodiment of the present application. As shown in FIG. 12, operation 603 includes: a step 1201 for controlling the image capture apparatus to acquire an image including the site to be examined and step 1202 for determining a position of the site to be examined in the image. Operation 603 further includes a step 1203 for determining, according to the selected image capture apparatus parameter and the position of the site to be examined in the image, a distance to be moved by a table carrying the site to be examined, wherein after the table is moved by the distance, the site to be examined is aligned with a scan center of the medical imaging system.

In some embodiments, in 1201 and 1202, the image capture apparatus is controlled to acquire an image I″ including the site to be examined, and the image I″ acquired by the image capture apparatus is acquired. A coordinate X″ of the pixel coordinate system corresponding to the site to be examined is determined in the image I″. For example, the site to be examined in the image is identified by using a deep learning algorithm or a machine learning algorithm, and the coordinate X″ of the site to be examined is determined. Image data of a plurality of volunteers located on the table may be acquired in advance as an input parameter set, and coordinates of a site to be examined corresponding to the image data that are calibrated in advance may be used as an output parameter set. The deep learning algorithm is trained by using the input parameter set and the output parameter set. The coordinate of the site to be examined is determined by using the trained deep learning algorithm.

In some embodiments, in 1203, after a distance Z (in the pixel coordinate system) that needs to be moved by the site to be examined is estimated according to the type and the coordinate position of the site to be examined by using the deep learning algorithm or the machine learning algorithm, alignment with the scan center is performed. The type and coordinate positions of sites to be examined of a plurality of volunteers located on the table may be acquired in advance as an input parameter set, and the movement distance that corresponds to the type and the coordinate of each site to be examined and that is measured or calculated in advance is used as an output parameter set. The deep learning algorithm is trained by using the input parameter set and the output parameter set. The distance Z that needs to be moved by the site to be examined is determined by using the trained deep learning algorithm.

In some embodiments, a distance Z′ to be moved by the table carrying the site to be examined may be calculated according to the image capture apparatus parameter M″ and the distance Z in combination with the internal parameters and the external parameters of the image capture apparatus. The distance Z is a distance in the medical imaging coordinate system (that is, the distance Z′ that needs to be moved in order to position the site to be examined in the medical imaging system coordinate system). For example, the distance Z′ in the medical imaging coordinate system is calculated by inputting the internal parameter (substituting for

$$[ \begin{matrix}{f}_{x} & 0 & {u}_{0} & 0 \\ 0 & {f}_{y} & {v}_{0} & 0 \\ 0 & 0 & 1 & 0\end{matrix} ]$$

in formula (6)) of the image capture apparatus, the image capture apparatus parameter M″ (substituting for

$$[ \begin{matrix}R & t \\ 0 & 1\end{matrix} ]$$

in formula (6)), and the distance Z (substituting for the coordinate distance in the pixel coordinate system in formula (6)) into formula (6).

It should be noted that FIGS. 6, 7, 10 to 12, and 15 above merely schematically illustrate the embodiments of the present application, but the present application is not limited thereto. For example, the order of execution between operations may be appropriately adjusted. In addition, some other operations may be added or some operations may be omitted. Those skilled in the art can make appropriate modifications according to the above content, rather than being limited by the descriptions of FIGS. 6, 7, 10 to 12, and 15.

Via the above embodiments, a plurality of image capture apparatus parameters corresponding to a plurality of regions in the medical imaging system are determined, and one image capture apparatus parameter is selected from the plurality of image capture apparatus parameters to perform coordinate conversion, so that image capture apparatus calibration precision in all positions on a table can be ensured, thereby improving accuracy of automatic positioning prediction. In addition, the entire calibration procedure is automatically performed, thereby avoiding human operation errors and simplifying the scanning procedure.

In addition, the table is controlled to sequentially move to a plurality of calibration positions corresponding to the plurality of regions, and images including a calibration tool on the table are acquired in the plurality of calibration positions, so as to determine the plurality of image capture apparatus parameters, so that system automation is high.

In addition, an initial calibration position for movement of the table is determined according to a maximum distance that the table can move and a distance between a positioning light and a scan center in the medical imaging system, and the calibration tool on the table is aligned with the positioning light, so that the plurality of image capture apparatus parameters corresponding to the plurality of regions can be acquired after the table is moved in the same direction once. The plurality of image capture apparatus parameters can cover all the regions of the table.

In addition, calibration accuracy can be improved by providing the checking step. Automated checking workflow can save a checking time.

The embodiments of the present application further provide a medical imaging system. FIG. 13 is a schematic diagram of a medical imaging system according to an embodiment of the present application. As shown in FIG. 13, the system 1300 includes: an image capture apparatus 1301, for capturing image data; and a controller 1302, connected to the image capture apparatus 1301, and configured to: determine a plurality of image capture apparatus parameters corresponding to a plurality of regions in the medical imaging system; select, from the plurality of image capture apparatus parameters, an image capture apparatus parameter corresponding to a region in which a site to be examined of a subject under examination is located; and perform processing of positioning of the site to be examined according to the selected image capture apparatus parameter.

In some embodiments, the medical imaging system further includes a movable table 1303. The controller 1302 is connected to the table 1303, and controls movement of the table. For example, when M image capture apparatus parameters are determined in advance, the controller 1302 controls the table 1303 to sequentially move to a plurality of calibration positions. In the plurality of calibration positions, the image capture apparatus 1301 is controlled to respectively acquire images including a calibration tool on the table. The controller 1302 acquires the image acquired by the image capture apparatus (for example, the image capture apparatus sends the acquired image to the controller 1302), and determines the plurality of image capture apparatus parameters according to the acquired images. For specific implementations, reference may be made to the foregoing embodiments, and details will not be described herein again. The above steps performed by the controller 1302 are all controlled by a program, so that an entire calibration procedure is automatically performed, thereby avoiding human operation errors.

Regarding implementations of the image capture apparatus 1301 and the controller 1302, reference may be made to the foregoing embodiments, which will not be repeated herein. The medical imaging system includes, but is not limited to: a computed tomography (CT) system, a magnetic resonance imaging (MRI) system, a C-arm imaging system, a positron emission computed tomography (PET) system, a single photon emission computed tomography (SPECT) system, an ultrasonic system, an X-ray imaging system, or any other suitable medical imaging system.

In some examples, the controller 1302 may be configured separately from a controller of the medical imaging system. For example, the controller 1302 is configured as a chip or the like connected to the controller of the medical imaging system, and the two controllers may control each other. Alternatively, functions of the controller 1302 may also be integrated into the controller of the medical imaging system. Embodiments of the present application are not limited thereto.

In some examples, the controller 1302 includes a computer processor and a storage medium. Recorded on the storage medium is a program for predetermined data processing to be executed by the computer processor. For example, stored on the storage medium may be a program for performing positioning assisting for an image capture apparatus. The storage medium may include, for example, a ROM, a floppy disk, a hard disk, an optical disk, a magneto-optical disk, a CD-ROM, or a non-volatile memory card.

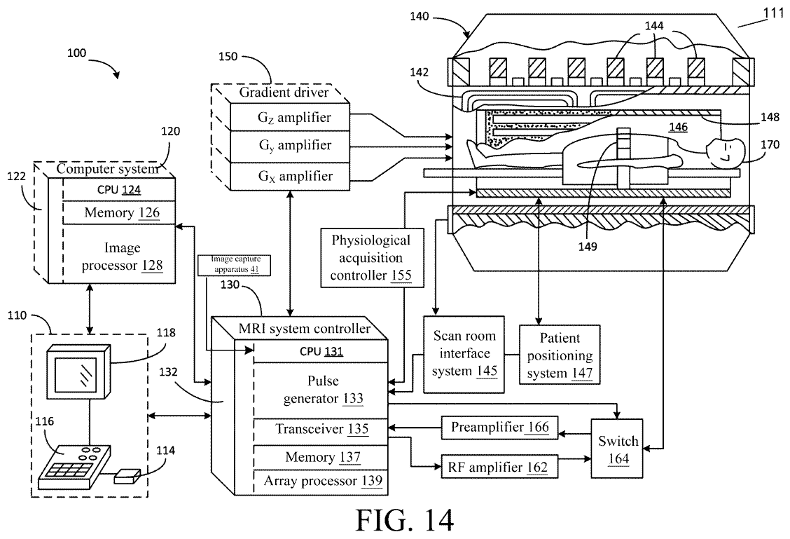

The medical imaging system may further include other structural components not shown in the drawing. For details, reference may be made to the related art, and the embodiments of the present application are not limited thereto. Taking a magnetic resonance imaging system as an example, FIG. 14 is a schematic diagram of a magnetic resonance imaging system according to an embodiment of the present application. As shown in FIG. 14, the difference from FIG. 5 is that the magnetic resonance imaging system further includes an image capture apparatus 41 that captures image data. In addition, a controller 1302 or a computer system 120 of the magnetic resonance imaging system is connected to the image capture apparatus 41 and is configured to: determine a plurality of image capture apparatus parameters corresponding to a plurality of regions in the medical imaging system; select, from the plurality of image capture apparatus parameters, an image capture apparatus parameter corresponding to a region in which a site to be examined of a subject under examination is located; and perform processing of positioning of the site to be examined according to the selected image capture apparatus parameter. For specific implementations thereof, reference may be made to the foregoing embodiments, which will not be repeated herein.

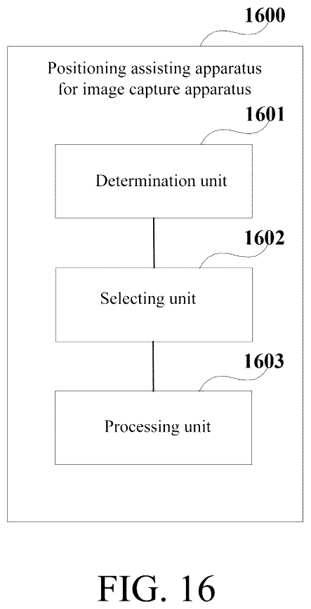

The embodiments of the present application further provide a positioning assisting apparatus for an image capture apparatus. FIG. 16 is a schematic diagram of a positioning assisting apparatus for an image capture apparatus according to an embodiment of the present application. As shown in FIG. 16, the apparatus 1600 includes: a determination unit 1601, for determining a plurality of image capture apparatus parameters corresponding to a plurality of regions in the medical imaging system; a selecting unit 1602, for selecting, from the plurality of image capture apparatus parameters, an image capture apparatus parameter corresponding to a region in which a site to be examined of a subject under examination is located. The apparatus 1600 further includes a processing unit 1603, for performing processing of positioning of the site to be examined according to the selected image capture apparatus parameter.

For implementations of the foregoing units and modules, reference may be made to 601 to 603, and the repeated description will not be provided again.

The embodiments of the present application further provide a computer-readable program, where the program, when executed in an apparatus or a medical imaging system, causes a computer to execute, in the apparatus or the medical imaging system, the positioning assisting method for an image capture apparatus according to the foregoing embodiments.

The embodiments of the present application further provide a storage medium having a computer-readable program stored therein, where the computer-readable program causes a computer to execute, in an apparatus or a medical imaging system, the positioning assisting method for an image capture apparatus according to the foregoing embodiments.

The embodiments of the present application further provide a computer program product at least including a computer program/instruction, where the computer program/instruction, when executed by a processor, perform the positioning assisting method for an image capture apparatus according to the foregoing embodiments.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A positioning assisting method for an image capture apparatus, applied to a medical imaging system, characterized by comprising:
determining a plurality of image capture apparatus parameters corresponding to a plurality of regions in the medical imaging system;
selecting, from the plurality of image capture apparatus parameters, an image capture apparatus parameter corresponding to a region in which a site to be examined of a subject under examination is located; and
performing processing of positioning of the site to be examined according to the selected image capture apparatus parameter.

2. The method according to claim 1, wherein the image capture apparatus parameter comprises a conversion matrix between an image capture apparatus coordinate system and a medical imaging system coordinate system.

3. The method according to claim 1, wherein the determining a plurality of image capture apparatus parameters corresponding to a plurality of regions in the medical imaging system comprises:
controlling a table of the medical imaging system to sequentially move to a plurality of calibration positions corresponding to the plurality of regions;
in the plurality of calibration positions, controlling the image capture apparatus to respectively acquire images comprising a calibration tool on the table; and
determining the plurality of image capture apparatus parameters according to the acquired images.

4. The method according to claim 3, further comprising:
determining an initial calibration position for movement of the table according to a maximum distance that the table can move and a distance between a positioning light and a scan center in the medical imaging system, controlling the table to move to the initial calibration position, and aligning the calibration tool on the table with the positioning light.

5. The method according to claim 3, wherein a distance of each movement of the table and the number of calibration positions are determined according to a size of the calibration tool and a length of the table.

6. The method according to claim 3, further comprising:
controlling the table to move to a checking position;
estimating a position of the calibration tool in a pixel coordinate system according to an image capture apparatus parameter corresponding to a region in which the checking position is located; and
comparing the estimated position of the calibration tool in the pixel coordinate system with a position of the calibration tool in an image acquired by the image capture apparatus in the checking position, and determining a checking result according to a comparison result.

7. The method according to claim 1, wherein the selecting, from the plurality of image capture apparatus parameters, an image capture apparatus parameter corresponding to a region in which a site to be examined of a subject under examination is located comprises:
determining a position of the site to be examined according to a position of the site to be examined relative to a table of the medical imaging system and a position of the table;
determining, from the plurality of regions, a region in which the position of the site to be examined is located; and
selecting, from the plurality of image capture apparatus parameters, an image capture apparatus parameter corresponding to the region.

8. The method according to claim 1, wherein the performing processing of positioning of the site to be examined according to the selected image capture apparatus parameter comprises:
controlling the image capture apparatus to acquire an image comprising the site to be examined;
determining a position of the site to be examined in the image; and
determining, according to the selected image capture apparatus parameter and the position of the site to be examined in the image, a distance to be moved by a table carrying the site to be examined, wherein after the table is moved by the distance, the site to be examined is aligned with a scan center of the medical imaging system.

9. A computer-readable storage medium, comprising a stored computer program, wherein the positioning assisting method for an image capture apparatus according claim 1 is performed when the computer program is run.

10. A medical imaging system, characterized by comprising:
an image capture apparatus, for capturing image data; and
a controller, connected to the image capture apparatus and configured to perform the positioning assisting method for an image capture apparatus according to claim 1.
